(19) United States
(12) Patent Application Publication
(10) Pub. No.: US 2015/0004229 A1
(43) Pub. Date: Jan. 1, 2015

(54) COMBINED PHARMACEUTICAL FORMULATION CONTAINING DIACEREIN

(71) Applicant: Sanovel Ilac Sanayi Ve Ticaret Anonim Sirketi, Istanbul (TR)

(72) Inventors: Umit Cifter, Istanbul (TR); Ali Turkyilmaz, Istanbul (TR); Onur Mutlu, Istanbul (TR)

(21) Appl. No.: 14/365,205

(22) PCT Filed: Dec. 25, 2012
(86) PCT No.: PCT/TR2012/000246

§ 371 Date: Jun. 13, 2014

(30) Foreign Application Priority Data
Dec. 27, 2011 (TR) 2011-13006
Aug. 1, 2012 (TR) 2012-08987

(51) Int. Cl. A61K 9/24; A61K 31/222; A61K 9/20; A61K 31/192

(52) U.S. Cl. CPC A61K 9/209; A61K 31/192; A61K 31/222; A61K 9/2018; A61K 9/2054; A61K 9/2009; A61K 9/2013; A61K 9/2027
USPC 424/465; 514/510

(57) ABSTRACT

The present invention relates to a combined pharmaceutical formulation having therapeutic anti-inflammatory, analgesic, antipyretic and osteoarthritis activities. From another perspective, the present invention relates to a new solid oral dosage form comprising a pharmaceutically effective amount of NSAID and a pharmaceutically effective amount of diacerein and the solid oral dosage form according to the present invention is preferably in the form of tablet and more preferably in the form of multi-layer tablet.

### TECHNICAL FIELD

The present invention relates to a new solid oral dosage form comprising a pharmaceutically effective amount of NSAID and a pharmaceutically effective amount of diacerein and the solid oral dosage form according to the present invention is preferably in the form of tablet and more preferably in the form of multi-layer tablet.

### BACKGROUND OF THE INVENTION

Non-steroidal anti-inflammatory drugs (NSAIDs) are drugs with analgesic, anti-inflammatory, antipyretic and osteoarthritis therapeutic activities and therefore are frequently used. NSAIDs are well defined group of compounds and comprise molecules derivatives of salicylate, acetic acid, propionic acid. Propionic acid derivative NSAIDs can be given as ibuprofen, naproxen, ketoprofen, fenoprofen, benoxaprofen, suprofen, flurbiprofen, ibuproxam, alminoprofen, dexibuprofen, dexketoprofen and indoprofen. These molecules are used in the treatment or prevention of symptoms such as pain, inflammation and fever. These symptoms also include those of musculoskeletal and joint disorders such as ankylosing spondylitis, osteoarthritis and rheumatoid arthritis, soft tissue injuries such as sprains and strains, post-operative pain, dolorous and difficult menstruation and migraine pains.

Another molecule that can be used especially in the treatment of osteoarthritis is diacerein disclosed in the U.S. Pat. No. 4,244,968. Said molecule is interleukin-1 inhibitor having anthraquinone structure and chemical structure thereof is shown in Formula 1.

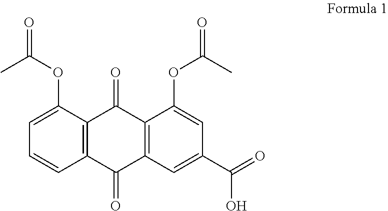 [SMILES: CC(=O)Oc1cccc2c1C(=O)c1c(OC(C)=O)cc(C(=O)O)cc1C2=O]

Use of NSAIDs and diacerein may cause some, particularly gastrointestinal, side effects. The most common side effect of NSAIDs is defined as a burning sensation in the gastrointestinal system. Diacerein, on the other hand, shows gastrointestinal side effect of diarrhea. Avoiding systemic adverse effects of NSAIDs and diacerein is very important for the patient. To prevent these side effects, various coating processes and formulations with different release profiles have been developed. For example, flurbiprofen is enterically coated in order to minimize damage to the stomach (Hashmat D., Shoaib H., Mehmood Z. (2008) AAPS PharmaSci Tech 9(1): 116-121) or formulated so as to provide controlled release (EP0234670). Furthermore, as described in the patent numbered EP1169032B1, ketoprofen, which is another propionic acid derivative, is coated with a polymeric layer providing sustained release. Side effects of diacerein is tried to be minimized by formulating it in hydrogel matrix (EP0809995). NSAIDs and diacerein are molecules with poor solubility in water. If a molecule has poor water solubility; absorption and hence bioavailability of said molecule are low. Thus, slow and late release of NSAIDs and diacerein, having already poor solubility, from the formulations developed in the prior art decreases the bioavailability of these two molecules.

Diacerein is available in the market under the brand names such as Artrodar®, Rexena® and is in the form of capsule. As is known, diacerein has a different mechanism of action than the other NSAIDs, however the use thereof with other NSAIDs can cause an additional effect. For this reason, today, although an oral pharmaceutical formulation comprising combination of diacerein and NSAID doesn't exist, these two molecules are used together, especially in the treatment of osteoarthritis. However, caution must be taken when using said two molecules simultaneously considering the side effects that can be caused by them. In addition, lack of a formulation comprising the two molecules together causes the patients to carry more than one drug and difficulty in administration of said drugs. Apart from that, another drawback is the intake of excipients in excess amount resulting from the intake of more than one drug, because excipients can also cause side effects as the active ingredients.

There are many points to be considered while formulating two active ingredients in a pharmaceutical formulation. These active ingredients should not be adversely affected from each other, at the same time, the two active ingredients should be released from the formulation in a concordant and coordinated manner. In addition, NSAIDs and diacerein, as in the case of many active ingredients, show stability problems resulting from the influence of the environmental and physical conditions. They are affected greatly by the temperature, light, air and humidity conditions; additionally, they are also affected by the solvents used during formulation thereof. Exposure to air and moisture causes structural destruction of said active ingredients and leads to the development of chemical behavior change. The stability of the developed products is not at a desired level and the shelf life thereof is shortened.

Consequently, stable pharmaceutical formulations with therapeutic analgesic, antipyretic and osteoarthritis activities, where NSAID and diacerein can be formulated together are still needed.

### OBJECTS OF THE INVENTION

The object of the present invention is to obtain a combined pharmaceutical formulation comprising an NSAID and diacerein, having therapeutic analgesic, antipyretic and/or osteoarthritis activities, eliminating all the aforementioned drawbacks and providing additional advantages to the respective technical field.

Another object of the present invention is to increase the effectiveness of the patient's diacerein treatment by administering NSAID to the patient being treated continuously with diacerein.

Another object of the present invention is to obtain a pharmaceutical formulation providing anti-inflammatory, analgesic, antipyretic and/or osteoarthritis treatment wherein NSAID and diacerein molecules do not adversely affect each other physically and chemically.

Another object of the present invention is to obtain a pharmaceutical formulation comprising a pharmaceutically effective amount of NSAID and a pharmaceutically effective amount of diacerein with desired level of solubility and dissolution rate of said two molecules, thus, with desired level of bioavailability and being released quickly.

Another object of the present invention is to obtain a combined pharmaceutical formulation comprising a pharmaceutically effective amount of NSAID and a pharmaceutically effective amount of diacerein with said two molecules being released in a coordinated manner.

Another object of the present invention is to obtain a pharmaceutical formulation comprising an NSAID and diacerein, having therapeutic anti-inflammatory, analgesic, antipyretic and/or osteoarthritis activities with gastrointestinal side effects being minimized.

Another object of the present invention is to decrease, by means of a unit dosage form comprising NSAID and diacerein, the amount of excipients (e.g., lactose) that could especially cause side effects when said molecules are administered individually.

Another object of the present invention is to obtain a stable unit dosage form comprising a pharmaceutically effective amount of NSAID together with a pharmaceutically effective amount of diacerein and at least one pharmaceutically acceptable excipient. Accordingly, the object of the present invention is to select the suitable excipient composition for providing the stability within the tablet.

### DESCRIPTION OF THE FIGURES

FIG. 1 shows a solid multi-layer oral tablet formulation comprising the following: i) a first layer containing the first active agent (a); ii) a second layer containing the second active agent (c); and iii) a barrier layer separating said two layers (b).

### DETAILED DESCRIPTION OF THE INVENTION

The pharmaceutical formulation according to the present invention is effective in prevention and reduction and/or treatment of symptoms such as pain, inflammation and/or fever and osteoarthritis wherein said composition comprises combination of an NSAID and diacerein. These symptoms also include those of musculoskeletal and joint disorders such as ankylosing spondylitis, osteoarthritis and rheumatoid arthritis, soft tissue injuries such as sprains and strains, post-operative pain, dolorous and difficult menstruation migraine pains and all forms of headaches.

The present invention relates to a new solid oral dosage form comprising a pharmaceutically effective amount of NSAID and a pharmaceutically effective amount of diacerein. The solid oral dosage form according to the present invention is preferably in the form of tablet and more preferably in the form of multi-layer tablet.

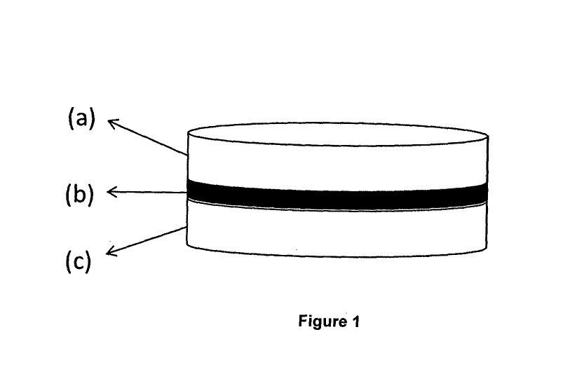

The present invention relates to a solid oral multi-layer tablet formulation comprising the followings: i) a first layer containing an NSAID molecule as the first active agent and pharmaceutically acceptable excipient or excipients; ii) a second layer containing diacerein molecule as the second active agent and pharmaceutically acceptable excipient or excipients; and iii) a barrier layer positioned between said two layers and containing pharmaceutically acceptable excipient or excipients (FIG. 1). In the tablet, these 3 layers are provided in the form of sandwich. Barrier layer is provided in the intermediate portion so as to separate NSAID and diacerein layers from each other and NSAID and diacerein layers are provided on both sides of the barrier layer on the outside portion. Negative interaction of NSAID and diacerein molecules are prevented by means of the barrier layer provided in the intermediate portion.

The NSAID in the multi-layer tablet formulation according to the present invention is a propionic acid derivative wherein it can be given as ibuprofen, naproxen, ketoprofen, fenoprofen, benoxaprofen, suprofen, flurbiprofen, ibuproxam, alminoprofen, dexibuprofen, dexketoprofen, indoprofen and mixture thereof and preferably said NSAID is flurbiprofen, dexketoprofen and mixture thereof.

On the other hand, NSAID and diacerein in the multi-layer tablet formulation according to the present invention can be present as pharmaceutically acceptable salts, enantiomers, racemates, polymorphs, esters and/or hydrates thereof.

NSAID group molecules and diacerein are molecules wherein its absorption occurs in the gastrointestinal system. The multi-layer tablet according to the present invention, as per tablet shape, provides disintegration of both molecule layers when taken into the body. NSAID and diacerein layers starts to disintegrate when taken into the body as it doesn't contain any coating or top layer. Resulting from said disintegration, NSAID and diacerein molecules solubilizes. Thus, formulation of said molecules having very low solubility in the multi-layer tablet according to the present invention positively affects the dissolution profiles of said molecules.

Another positive effect of the multi-layer tablet formulation according to the present invention is the prevention of adverse physical and chemical interaction of NSAID and diacerein molecules, provided in two separate layers and thus, a more stable formulation is provided.

The pharmaceutically acceptable excipients provided in the multi-layer tablet formulation according to the present invention can be selected from, but not limited to, diluents, binders, disintegrants, glidants, lubricants, plasticizers, surfactants, preservatives or mixtures thereof.

Diluents according to the present invention include, but not limited to, lactose, microcrystalline cellulose, corn starch, modified corn starch, calcium phosphate, sugar, dextrose, mannitol, sorbitol, starch, pregelatinized starch and mixtures thereof.

Binders according to the present invention include, but not limited to, lactose, polymethacrylate, polyvinylpyrrolidone (povidone), hydroxypropyl methyl cellulose (HPMC), hydroxypropyl cellulose (HPC), carboxymethyl cellulose (CMC), methyl cellulose (MC), hydroxyethyl cellulose, sodium carboxy methyl cellulose (NaCMC), carboxymethyl cellulose calcium, ethyl cellulose, polyethylene oxide, gelatin, starch, pregelatinized starch, xanthan gum, guar gum, alginate, carrageenan, pectin, carbomer, cellulose acetate phthalate, hydroxy propyl starch, polaxomer, polyethylene glycol and mixtures thereof.

Disintegrants according to the present invention include, but not limited to, microcrystalline cellulose, croscarmellose sodium, xylitol, polyplasdone (1-ethenylpyrrolidin-2-one), crospovidone, hydroxypropyl cellulose, low-substituted hydroxypropyl cellulose (L-HPC) and sodium starch glycolate or mixtures thereof.

Glidants according to the present invention include, but not limited to, silicon dioxide, magnesium trisilicate, starch, talc, colloidal silicon dioxide or silicon hydrogel or mixtures thereof.

Lubricants according to the present invention include, but not limited to, magnesium stearate, sodium stearyl fumarate, polyethylene glycol, stearic acid, metal stearates, boric acid, sodium chloride benzoate and acetate, sodium or magnesium lauryl sulfate or mixtures thereof.

Plasticizers according to the present invention include, but not limited to, triethyl citrate, triacetin, citric acid esters, phthalic acid esters, dibutyl sebacate, cetyl alcohol, polyethylene glycol, polysorbate or mixtures thereof.

Surfactants according to the present invention may include, but not limited to, dioctyl sulfosuccinate, polysorbates and polyoxyethylene alkyl esters and ethers thereof, glyceryl monolaurate saponins, sorbitan laurate, sodium lauryl sulfate, magnesium lauryl sulfate or mixtures thereof.

Preservatives according to the present invention include, but not limited to, methyl paraben, propyl paraben and salts thereof (e.g. sodium or potassium salts), sodium benzoate, citric acid, benzoic acid, butylated hydroxytoluene and butylated hydroxyanisole or mixtures thereof.

The present invention, in a preferred embodiment thereof, also include at least one or a mixture in the suitable amount of polyvinyl alcohol-polyethylene glycol copolymer, polyoxyethylene-polyoxypropylene block copolymer, stearyl macrogol glyceride, polyethylene glycol, povidone, cationic methacrylate, copovidone, derivatives of methacrylic acid copolymer, cellulose acetate phthalate, acetylated monoglyceride, dibutyl tartrate, diethyl phthalate, dimethyl phthalate, glycerine, propylene glycol, stearic acid, triacetin, triacetin citrate and tripropionin.

Excipients comprised within the content of the multi-layer tablet according to the present invention is selected and formulated so as to optimize disintegration rate of said tablet. In the formulation according to the present invention, disintegrant agent provided in each layer containing the active ingredients is in the range of 0.5% to 30% by weight of the layer. With disintegrant agent present in such amount, formulation gains the hardness required for the sandwich form as well as disintegrates quickly.

According to another embodiment of the present invention, NSAID and diacerein layers comprise croscarmellose sodium and hydroxypropyl cellulose as disintegrant. Said disintegrant agents swell by absorbing the water upon contact therewith and provide rapid disintegration of the tablet. It has been found out that the first and second layers comprising active ingredient provided in the multi-layer tablet formulation according to the present invention with croscarmellose sodium agent about 3% to 7% by weight of the layer and hydroxypropyl cellulose agent about 2% to 10% by weight of the layer causes synergistic effect on the disintegration time. Higher amounts can lead to adverse effects on the mechanical resistance of the formula while lower amounts can worsen the disintegration time. In said ranges, layers containing croscarmellose sodium and hydroxypropyl cellulose disintegrate rapidly as well as they are hard enough to take the multi-layer tablet form according to the present invention.

According to another embodiment, the present invention concerns inclusion of glidant in the layers of the multi-layer tablet formulation comprising NSAID and diacerein wherein said glidant is preferably colloidal silicon dioxide. Colloidal silicon dioxide provided in the layers is in the range of approximately 0.01% to 1% by weight of the layers. It has been found out that the effectiveness of the disintegrant agents increase with the colloidal silicon dioxide provided in said range in each layer. Colloidal silicon dioxide is a hydrophobic substance. Thus, large amount of said substance being present delays the contact of the disintegrant agents with water and leads to delayed disintegration of the tablet.

According to another embodiment, the present invention concerns multi-layer tablet formulation comprising NSAID and diacerein wherein lactose and derivatives thereof are comprised therein as excipient. Total lactose amount provided in the tablet according to the present invention is in the range of 14% to 43% by weight of total tablet weight. Thus, potential side effects that may be observed in the patient (e.g., lactose intolerance) are reduced. When NSAID and diacerein preparations available in the prior art are used separately, total lactose intake of the patient increases. When these two molecules are brought together in the form of multi-layer tablet, lactose intake decreases in the range of approximately 30% to 60% and thus, potential side effects that can be observed in the patient reduces.

According to another embodiment, in the multi-layer tablet formulation according to the present invention, lactose amount comprised in the layer containing NSAID is in the range of 7% to 43% by weight of respective layer and lactose amount comprised in the layer containing diacerein is in the range of 50% to 60% by weight of respective layer. With lactose being present in said ranges, multi-layer tablet is not affected by environmental conditions such as humidity in particular and becomes more stable. In addition, compressibility property of the tablet is at the optimum level with lactose provided in such ranges.

Pharmaceutically acceptable lactose according to the present invention is selected from the group comprising spray-dried lactose, beta-lactose, alpha lactose monohydrate, lactose anhydride or mixtures thereof, preferably, said lactose is spray-dried lactose.

The multi-layer tablet according to the present invention comprises pharmaceutically acceptable lactose having 98% of particles thereof with size in the range of approximately 300 μm to 350 μm.

From another perspective, the amount of NSAID group molecules provided in the multi-layer tablet formulation according to the present invention is in the range of 5 mg to 1000 mg and preferably, among said molecules, flurbiprofen is provided in the range of approximately 50 mg to 300 mg and dexketoprofen is provided in the range of approximately 5 mg to 100 mg and more preferably, flurbiprofen amount is 100 mg or 50 mg and dexketoprofen amount is 25 mg.

From another perspective, the amount of diacerein provided in the multi-layer tablet formulation according to the present invention is in the range of approximately 10 mg to 100 mg and preferably, diacerein amount is 50 mg.

The present invention concerns a multi-layer tablet comprising NSAID layer wherein it includes, but may not be limited to, the following substances by weight of the respective layer:
a) approximately 5% to 60% by weight of NSAID,
b) approximately 5% to 60% by weight of diluent,
c) approximately 0.5% to 30% by weight of disintegrant,
d) approximately 2% to 10% by weight of binder,
e) approximately 0.1% to 10% by weight of glidant,
f) approximately 0.1% to 10% by weight of lubricant.

The present invention concerns a multi-layer tablet comprising diacerein layer wherein it includes, but may not be limited to, the following substances by weight of the respective layer:
a) approximately 5% to 50% by weight of diacerein,
b) approximately 30% to 75% by weight of diluent,
c) approximately 0.5% to 30% by weight of disintegrant.
d) approximately 1% to 10% by weight of binder,
e) approximately 0.01% to 5% by weight of glidant,
f) approximately 1% to 10% by weight of lubricant.

From another perspective, the present invention concerns a multi-layer tablet in the form of sandwich comprising a barrier layer separating a first layer containing NSAID and a second layer containing diacerein wherein the barrier layer constitutes the middle layer of the multi-layer tablet and comprises pharmaceutically suitable excipients. Incompatibility of NSAID and diacerein molecules within the formulation is minimized or prevented by means of said barrier layer.

The pharmaceutically acceptable excipients provided in the barrier layer according to the present invention include binders, disintegrants, glidants, lubricants, plasticizers, surfactants, preservatives or mixtures thereof.

Multi-layer tablet formulation according to the present invention can be prepared by various conventional methods known in the respective technical field. Said methods can be given as, but not limited to, wet granulation, dry granulation, direct compression, melt granulation and double compression.

NSAID containing layer provided in the multi-layer tablet formulation according to the present invention can be prepared by various conventional methods known in the respective technical field wherein it is preferably prepared by wet granulation or dry granulation methods. Thus, potential flowability problems that can be encountered with NSAID molecules are resolved.

Diacerein containing layer provided in the multi-layer tablet formulation according to the present invention can be prepared by various conventional methods known in the respective technical field wherein it is preferably prepared by dry granulation or direct compression methods.

Barrier layer provided in the multi-layer tablet formulation according to the present invention can be prepared by various conventional methods known in the respective technical field wherein it is preferably prepared by direct compression method.

Finally, 3 layers obtained by methods according to the present invention are compressed together so as to form a tablet where the barrier layer is positioned in the middle.

The term formulation may refer to formulation as well as both the packaging or blisters in which the formulation is stored. Multi-layer tablet formulation according to the present invention is preferably stored in opaque PVC/PVDC packaging and the formulation thus becomes more stable. The main reason for this is the light sensitivity of the diacerein molecule.

### EXAMPLES

The following examples show the preferred oral pharmaceutical formulations of the present invention.

### Example 1

Multi-layer tablet formulation, manufacturing method of which is described below, contains 100 mg flurbiprofen and 50 mg diacerein.

| | | Amount per tablet |
|---|---|---|
| | Constituent | (mg) |
| Layer I | Flurbiprofen | 100.00 |
| | Lactose | 32.60 |
| | Microcrystalline cellulose | 132.00 |
| | Croscarmellose sodium | 14.00 |
| | Hydroxypropyl cellulose | 18.50 |
| | Colloidal silicon dioxide | 1.90 |
| | Magnesium stearate | 1.00 |
| | First layer total | 300.00 |
| Intermediate | Sodium | 44.70 |
| Layer | carboxymethylcellulose | |
| | Polyvinylpyrrolidone | 6.00 |
| | Microcrystalline cellulose | 22.1 |
| | Yellow iron oxide | 0.30 |
| | Colloidal silicon dioxide | 0.40 |
| | Magnesium stearate | 1.5 |
| | Intermediate layer total | 75.00 |
| Layer II | Diacerein | 50.00 |
| | Lactose | 83.40 |
| | Croscarmellose sodium | 7.50 |
| | Hydroxypropyl cellulose | 5.00 |
| | Colloidal silicon dioxide | 0.10 |
| | Sodium stearyl fumarate | 4.00 |
| | Second layer total | 150.00 |

Manufacturing Method:
a) Preparation of the first layer containing flurbiprofen is carried out as follows:
   i. Flurbiprofen, a portion of microcrystalline cellulose, lactose, croscarmellose sodium and hydroxypropyl cellulose are provided;
   ii. Colloidal silicon dioxide and rest of the microcrystalline cellulose are sieved, added into the mixture obtained in step i and mixed;
   iii. Tablets are compressed into bricks, crushed and sieved;
   iv. Magnesium stearate is added into the mixture obtained in step iii and mixed.
b) Preparation of the second layer containing diacerein is carried out as follows:
   i. Diacerein, a portion of lactose, croscarmellose sodium and hydroxypropyl cellulose are provided;
   ii. Colloidal silicon dioxide and rest of the lactose are sieved, added into the mixture obtained in step i and mixed;
   iii. Sodium stearyl fumarate is added into the mixture obtained in step ii and mixed.
c) Preparation of the barrier layer is carried out as follows:
   i. Sodium carboxymethyl cellulose, polyvinylpyrrolidone, a portion of microcrystalline cellulose and yellow iron oxide are provided;
   ii. Rest of the microcrystalline cellulose and colloidal silicon dioxide are sieved, added into the mixture obtained in step i and mixed;
   iii. Magnesium stearate is added into the mixture obtained in step ii and mixed.
d) Homogeneous mixtures obtained in steps a, b, and c are compressed so as to obtain a tablet in the form of sandwich with the barrier layer provided in the middle thereof.

### Example 2

Multi-layer tablet formulation, manufacturing method of which is described below, contains 100 mg flurbiprofen and 50 mg diacerein.

| | | Amount per tablet |
|---|---|---|
| | Constituent | (mg) |
| Layer I | Flurbiprofen | 100.00 |
| | Lactose | 124.50 |
| | Microcrystalline cellulose | 55.00 |
| | Croscarmellose sodium | 7.30 |
| | Hydroxypropyl cellulose | 9.80 |
| | Colloidal silicon dioxide | 2.10 |
| | Magnesium stearate | 1.30 |
| | First layer total | 300.00 |
| Layer II | Diacerein | 50.00 |
| | Lactose | 83.40 |
| | Croscarmellose sodium | 7.50 |
| | Hydroxypropyl cellulose | 5.00 |
| | Colloidal silicon dioxide | 0.10 |
| | Sodium Stearyl fumarate | 4.00 |
| | Intermediate layer total | 150.00 |
| Intermediate | Carboxymethyl cellulose | 44.70 |
| Layer | Polyvinil pyrollidone | 6.00 |
| | Microcrystalline cellulose | 22.1 |
| | Yellow Iron Oxide | 0.30 |
| | Colloidal silicon dioxide | 0.40 |
| | Magnesium Stearate | 1.5 |
| | Second layer total | 75.00 |

Manufacturing Method:
a) Preparation of the first layer containing flurbiprofen is carried out as follows:
   i. Flurbiprofen, microcrystalline cellulose, lactose and a portion of the hydroxypropyl cellulose are picked up and loaded into the fluidized bed dryer;
   ii. An aqueous solution is prepared with the rest of the hydroxypropyl cellulose;
   iii. Mixture obtained in step i. in the fluidized bed dryer is granulated with said solution, dried and milled;
   iv. Mixture obtained in step iii is transferred into the container;
   v. Croscarmellose sodium and colloidal silicon dioxide are added into the same container and mixed;
   vi. Magnesium stearate is added into the mixture obtained in step v and mixed.
b) Preparation of the second layer containing diacerein is carried out as follows:
   i. Diacerein, a portion of lactose, croscarmellose sodium and hydroxypropyl cellulose are mixed;
   ii. Colloidal silicon dioxide and rest of the lactose are sieved, added into the mixture obtained in step i and mixed;
   iii. Sodium stearyl fumarate is added into the mixture obtained in step ii and mixed.
c) Preparation of the barrier layer is carried out as follows:
   i. Sodium carboxymethyl cellulose, polyvinylpyrrolidone, a portion of microcrystalline cellulose and yellow iron oxide are mixed;
   ii. Rest of the microcrystalline cellulose and colloidal silicon dioxide are sieved, added into the mixture obtained in step i and mixed;
   iii. Magnesium stearate is added into the mixture obtained in step ii and mixed.
d) Homogeneous mixtures obtained in steps a, b, and c are compressed so as to obtain a tablet in the form of sandwich with the barrier layer provided in the middle thereof.

### Example 3

### Multi-Layer Tablet Formulation Containing Dexketoprofen and Diacerein

| Constituent | Amount per tablet (mg) |
|---|---|
| Dexketoprofen | 36.91 |
| trometamol (equivalent | |
| to 25 mg | |
| dexketoprofen) | |
| Lactose monohydrate | 138.6 |
| Corn starch | 65.0 |
| Sodium starch | 13.0 |
| glycolate | |
| Sodium stearyl | 6.5 |
| fumarate | |
| First layer total | 260.00 |
| Sodium | 44.70 |
| carboxymethylcellulose | |
| Polyvinylpyrrolidone | 6.00 |
| Microcrystalline | 22.1 |
| cellulose | |
| Yellow iron oxide | 0.30 |
| Colloidal silicon dioxide | 0.40 |
| Magnesium stearate | 1.5 |
| Intermediate layer | 75.00 |
| total | |
| Diacerein | 50.00 |
| Lactose | 83.40 |
| Croscarmellose sodium | 7.50 |
| Hydroxypropyl | 5.00 |
| cellulose | |
| Colloidal silicon dioxide | 0.10 |
| Sodium stearyl | 4.00 |
| fumarate | |
| Second layer total | 150.00 |

Manufacturing Method:
a) Preparation of the first layer containing dexketoprofen is carried out as follows:
   i. Dexketoprofen, lactose monohydrate and corn starch are picked up and loaded into the fluidized bed dryer;
   ii. Mixture obtained in step i in the fluidized bed dryer is granulated with water, dried and milled;
   iii. Mixture obtained in step ii is transferred into the container;
   iv. Sodium starch glycolate is added into the same contained and mixed;
   v. Sodium stearyl fumarate is added into the mixture obtained in step iv and mixed.
b) Preparation of the second layer containing diacerein is carried out as follows:
   i. Diacerein, a portion of lactose, croscarmellose sodium and hydroxypropyl cellulose are mixed;
   ii. Colloidal silicon dioxide and rest of the lactose are sieved, added into the mixture obtained in step i and mixed;
   iii. Sodium stearyl fumarate is added into the mixture obtained in step ii and mixed.
c) Preparation of the barrier layer is carried out as follows:
   i. Sodium carboxymethyl cellulose, polyvinylpyrrolidone, a portion of microcrystalline cellulose and yellow iron oxide are mixed;
   ii. Rest of the microcrystalline cellulose and colloidal silicon dioxide are sieved, added into the mixture obtained in step i and mixed;
   iii. Magnesium stearate is added into the mixture obtained in step ii and mixed.
d) Homogeneous mixtures obtained in steps a, b, and c are compressed so as to obtain a tablet in the form of sandwich with the barrier layer provided in the middle thereof.

1. A solid oral multi-layer tablet in the form of a sandwich, wherein it comprises:
i) a first layer containing a propionic acid derivative NSAID as the first active agent and pharmaceutically acceptable excipient or excipients;
ii) a second layer containing diacerein as the second active agent and pharmaceutically acceptable excipient or excipients; and
iii) a barrier layer positioned between these two layers and comprising pharmaceutically acceptable excipient or excipients.

2. A solid oral multi-layer tablet in the form of a sandwich according to claim 1, wherein the pharmaceutically acceptable excipient comprises at least one of binders, disintegrants, glidants, lubricants, plasticizers, surfactants, preservatives or mixtures thereof in a suitable amount.

3. A solid oral multi-layer tablet in the form of a sandwich according to claim 2, wherein the at least one of disintegrants is selected from microcrystalline cellulose, croscarmellose sodium, xylitol, polyplasdone (1-ethenylpyrrolidin-2-one), crospovidone, hydroxypropyl cellulose, low-substituted hydroxypropyl cellulose (L-HPC), sodium starch glycolate or mixtures thereof.

4. A solid oral multi-layer tablet in the form of a sandwich according to claim 3, wherein the at least one of disintegrants is microcrystalline cellulose or croscarmellose sodium.

5. A solid oral multi-layer tablet in the form of a sandwich according to claim 3, wherein the first and second layers comprise croscarmellose sodium in the range of 3% to 7% by weight in each of the first and second layers and hydroxypropyl cellulose agent in the range of 2% to 10% by weight of each of the first and second layers.

6. A solid oral multi-layer tablet in the form of a sandwich according to claim 2, wherein the at least one of glidants comprised therein is selected among silicon dioxide, magnesium trisilicate, starch, talc, colloidal silicon dioxide or silicon hydrogel or mixtures thereof.

7. A solid oral multi-layer tablet in the form of a sandwich according to claim 6, wherein the at least one of glidants comprised therein is colloidal silicon dioxide.

8. A solid oral multi-layer tablet in the form of a sandwich according to claim 7, wherein each layer comprises colloidal silicon dioxide in the range of 0.01% to 1% by weight of each of the respective layers.

9. A solid oral multi-layer tablet in the form of a sandwich according to claim 2, wherein the at least one of binders comprised therein is selected from lactose, polymethacrylate, polyvinylpyrrolidone (povidone), hydroxypropyl methyl cellulose (HPMC), hydroxypropyl cellulose (HPC), carboxymethyl cellulose (CMC), methyl cellulose (MC), hydroxy ethyl cellulose, sodium carboxy methyl cellulose (NaCMC), carboxymethyl cellulose calcium, ethyl cellulose, polyethylene oxide, gelatin, starch, pregelatinized starch, xanthan gum, guar gum, alginate, carrageenan, pectin, carbomer, cellulose acetate phthalate, hydroxy propyl starch, polaxomer, polyethylene glycol and mixtures thereof.

10. A solid oral multi-layer tablet in the form of a sandwich according to claim 9, wherein the at least one of binders comprised therein is lactose.

11. A solid oral multi-layer tablet in the form of a sandwich according to claim 10, wherein the amount of lactose comprised therein is in the range of 14% to 43% by weight of the total tablet.

12. A solid oral multi-layer tablet in the form of a sandwich according to claim 10, wherein the lactose comprised therein is spray-dried lactose.

13. A solid oral multi-layer tablet in the form of a sandwich according to claim 1, wherein the propionic acid derivative NSAID is selected from ibuprofen, naproxen, ketoprofen, fenoprofen, benoxaprofen, suprofen, flurbiprofen, ibuproxam, alminoprofen, dexibuprofen, dexketoprofen, indoprofen and mixtures thereof.

14. A solid oral multi-layer tablet in the form of a sandwich according to claim 13, wherein the propionic acid derivative NSAID is selected from flurbiprofen, dexketoprofen and mixtures thereof.
